(19) United States
(12) Patent Application Publication
(10) Pub. No.: US 2003/0082778 A1
(43) Pub. Date: May 1, 2003

(54) Regulation of promoter activity in cells

(76) Inventors: Soren Michael Madsen, Copenhagen N (DK); Astrid Vrang, Lyngby (DK); Lars Bredmose, Copenhagen (DK); Peter Ravn, Naerum (DK); Jacob Glenting, Copenhagen 0 (DK); Mads Gronvald Johnsen, Frederiksberg C (DK); Hans Israelsen, Allerod (DK)

Correspondence Address: Stanislaus Aksman, Hunton & Williams, Suite 1200, 1900 K Street, N.W., Washington, DC, 20006, US

(21) Appl. No.: 09/982,532

(22) Filed: Oct. 19, 2001

Related U.S. Application Data
(63) Continuation-in-part of application No. 09/692,204, filed on Oct. 20, 2000.

(51) Int. Cl. C12N 9/22; C07H 21/04; C12N 15/74; C12P 21/02; C12N 1/21

(52) U.S. Cl. 435/199; 435/69.1; 435/252.3; 435/320.1; 536/23.2

(57) ABSTRACT

Nucleotide sequences coding for a promoter activity-regulating polypeptide designated OrfY, isolated from Lactococcus lactis, and promoter activity-regulating active homologues and fragments hereof is used to indirectly regulating the expression of genes. The coding sequence is inserted into lactic acid bacterial cells or any other cells with the objective of either reducing or enhancing the expression level of the gene being under the control of the promoter sequence, the activity of which is regulated by the OrfY polypeptide or homologues or fragments hereof. Improved gene expression systems are provided based on this promoter activity-regulating polypeptide.

This application is a continuation in part of U.S. patent application No. 09/692,204 filed Oct. 20, 2000, entitled “Regulation of Promoter Activity in Cells,” the contents of which are incorporated herein in their entirety to the extent that it is consistent with this invention and application.

### FIELD OF INVENTION

In its broadest aspect, the present invention relates generally to the field of gene expression systems in microbial cells including lactic acid bacteria and in particular to regulatable expression systems that are useful in such bacteria intended for use as starter cultures in food and feed manufacturing or used as production strains in the manufacturing of desired gene products including pharmaceutically active substance such as vaccines. Specifically, the invention provides novel means of regulating the activity of regulatable (inducible) promoter sequences in such regulatable expression systems.

### TECHNICAL BACKGROUND AND PRIOR ART

For centuries, starter cultures of lactic acid bacteria have been used in food production due to their ability to convert sugars by fermentation into organic acids, predominantly lactic acid, and various metabolites associated with the development in the fermented food products of a desirable taste and flavour. In addition, several lactic acid bacteria inherently produce hydrolytic enzymes including peptidases, proteases and lipolytic enzymes, the production of which may e.g. contribute to a desired flavour development in cheeses. Based on their traditional and long term application in food manufacturing and the fact that they are considered as non-pathogenic, the lactic acid bacteria are generally recognised as safe (GRAS) food ingredients, even if they are present in a fermented food product as live bacteria at a very high number, such as 10⁸ to 10⁹ per g.

However, for industrial production of a wide range of fermented food products such as all the well-known traditional dairy products including yoghurt, acidophilus milk, butter and cheeses; fermented vegetables; fermented meat products and animal feed, a large range of lactic acid bacterial starter cultures, each being adapted to particular types of food products, are required. Such cultures are presently being selected from naturally occurring strains of lactic acid bacteria on the basis of characteristics such as their ability to ferment sugars present in the food product to be fermented, specific growth temperature requirements, production of desired flavouring compounds, the specific combination of which characteristics renders a specifically selected wild type culture useful for the production of a particular food product but normally less useful for the production of others.

Evidently, this conventionally used procedure for developing useful lactic acid bacterial cultures by selection of naturally occurring strains is cumbersome and costly. Furthermore, it has proven difficult to provide individual starter culture strains which combine all of the required characteristics at an optimal level. Previously, this problem has usually been solved by the use of starter cultures comprising a multiplicity of selected lactic acid bacterial strains each having one or several of the characteristics desirable for a particular food product. The necessity to use such mixed cultures will of course add to the costs in the manufacture of lactic acid bacterial starter cultures.

Currently, it is widely recognised that a substantial industrial need exists to find economically and technically more feasible ways of developing improved lactic acid bacteria for use as food or feed starter cultures or for the production of desired gene products including providing lactic acid bacteria which are useful for a wide range of applications. It is evident that recombinant DNA technology may provide the means to meet this need. In this context, it is crucial that lactic acid bacteria for food manufacturing which are developed by introduction of desired genes by use of gene technology can still be recognised as safe for consumption. It is therefore considered in the food industry that it may be advantageous that recombinant lactic acid bacteria essentially only contains DNA of lactic acid bacterial origin including DNA from wild type extrachromosomal plasmids frequently found in starter culture strains, or non-lactic acid bacterial DNA which does not confer any hazardous phenotypic traits to the recombinant strains.

There have been several attempts to provide genetically improved lactic acid bacteria. Most of these attempts have been directed to the construction of recombinant expression vectors coding for desired gene products and capable of replicating in lactic acid bacteria. However, very few of these attempts have resulted in vectors comprising only lactic acid bacterial DNA.

Inducible or regulatable gene expression systems are highly important for expression of genes encoding proteins that are either (i) toxic to the host organism, (ii) needed in large quantities, (iii) used to study the effect of particular gene functions on cellular metabolism or regulation or (iv) produced at a particular point in time or under particular environmental conditions. Whereas inducible expression systems have been developed for use in E. coli, only a few inducible expression systems for use in lactic acid bacteria have been described.

It has recently been discovered that it is possible to isolate lactic acid bacterial promoters which are inducible or regulatable by the presence/absence or the concentration of one or more environmental factors associated with conventional lactic acid bacterial industrial production methods such as pH, growth temperature, composition of the growth medium including the ionic strength/NaCI content, the presence/absence of purine nucleotide precursors, the accumulation intracellularly or in the medium of metabolites, and/or the growth phase/growth rate of the bacterium (WO 94/16086, Israelsen et al., 1995).

It is evident that regulatable expression systems based on such environmental or growth condition factors, which are normally present in industrial culture media for lactic acid bacteria, including starting materials for fermented products, either initially or during the culturing, represent a highly attractive approach for regulating the production of homologous or heterologous gene products in lactic acid bacteria. However, in order for the application of these regulatable expression systems to be successful, the selected promoter must be effective and its activity lead to the expression of desired gene products in sufficiently high amounts under industrial conditions to facilitate an economically viable production or manufacturing process. However, it has been found that such otherwise useful naturally occurring regulatable lactic acid bacterial promoter regions may only have a relatively weak promoter activity.

WO 98/10079 provides improved lactic acid bacterial regulatable gene expression systems which are based on improving the expression systems disclosed in WO 94/16086 by modification of the naturally occurring promoter regions of such systems which are operably associated or linked with a gene, whereby the expression of the gene can be enhanced significantly. This significant improvement was based on the discovery that the activity of naturally occurring inducible or regulatable lactic acid bacterial promoters can be increased significantly by modifying the nucleotide region in which the promoter is located and, most importantly, that such an increased promoter activity can be obtained without reducing or eliminating the inducibility conferred by the above growth condition factors. The achievements in WO 98/10079 also made it possible to provide sets or panels comprising lactic acid bacteria producing a desired gene product at different levels under identical conditions. Additionally, it was found in WO 98/10079 that modifications of the promoter region sequences may result in strains having a modulated expression level under induced conditions as compared to the regulation by the corresponding non-modified promoter region.

As used herein the expression “lactic acid bacterium” designates a gram-positive, microaerophilic or anaerobic bacterium which ferments sugars with the production of acids including lactic acid as the predominantly produced acid. The industrially most useful lactic acid bacteria are found among Lactococcus spp., Streptococcus spp., Lactobacillus spp., Leuconostoc spp., Oenococcus spp., Pediococcus spp., Brevibacterium spp. and Propionibacterium spp. Additionally, lactic acid producing bacteria belonging to the group of the strictly anaerobic bacteria, bifidobacteria, ie Bifidobacterium spp. which are frequently used as food starter cultures alone or in combination with lactic acid bacteria, are generally included in the group of lactic acid bacteria.

Lactococcus lactis is commonly used in the production of fermented dairy products such as cheese, sour cream and buttermilk. More recently, the advances in DNA technology have provided several opportunities for production of foods with new or improved properties (de Ruyter et al., 1997; Hols et al., 1999; Kleerebezem et al., 1999). Also, there has been an increasing focus on the exploitation of L. lactis as a cell factory for producing recombinant proteins (Kuipers et al., 1997) and the use of L. lactis as a vaccine delivery vehicle (Wells et al., 1996).

Successful expression of recombinant proteins is usually accomplished through the use of controlled or regulated gene expression systems. Therefore, a number of inducible promoters has been identified and exploited in gene expression systems for use in L. lactis and other lactic acid bacterial species (WO 98/10079; Kok, 1996; Djordjevic and Klaenhammer, 1998; Kuipers et al., 1997). The detailed knowledge of the regulatory elements and mechanisms that control the respective promoters is a prerequisite to achieve successful design of these systems.

A variety of regulating proteins that control promoter activity negatively or positively has been discovered and characterised at the molecular level (Kok, 1996). Some of these regulators are specific for a single gene/operon while others might be involved in control of several genes/operons. The molecular characterisation of the pH and growth

phase-dependent promoter P170 of L. lactis has been described in WO 98/10079 and Madsen et al., 1999. Using deletion analysis, the minimal DNA region required for both promoter activity and pH regulation has previously been mapped to a 51 bp region located 7 bp upstream of the transcriptional start site. The minimal promoter region contains an extended-10 promoter sequence but no consensus-35 sequence was identified. The lack of a consensus-35 region has also been observed in other L. lactis promoters (Sanders et al., 1998b; Walker and Klaenhammer, 1998) and usually induction of transcription requires a transcriptional activator.

By construction of hybrid promoters, a 27 bp DNA segment located 15 bp upstream of the extended-10 region of the P170 promoter that is responsible for the pH and growth phase regulated promoter activity has been identified. In the current model for promoter regulation, it is assumed that this 27 bp segment comprises a cis-acting site, which is recognised by a trans-acting regulatory factor for activity of inducible lactic acid bacterial promoters. The aims of the present invention were to identify and characterise this putative trans-acting factor and to possibly provide the basis for further improving the performance of inducible lactic acid bacterial gene expression systems.

As a result of the experimentation leading to the invention, a gene referred to herein as the orfY gene was identified in the chromosome of a Lactococcus lactis strain and it was found that inactivation of orfY resulted in a 100-fold reduction in the activity of a regulatable promoter. Furthermore, it has been shown that over-expression of orfY results in increased production of reporter gene products which is controlled by a regulatable promotor.

### SUMMARY OF THE INVENTION

Accordingly, the invention relates in a first aspect to an isolated nucleotide sequence comprising a coding sequence selected from the group consisting of (i) a sequence coding for the promoter activity-regulating polypeptide OrfY; (ii) a sequence coding for a polypeptide that is at least 15% identical to the sequence of (i) and that has at least part of the promoter activity-regulating activity of the OrfY polypeptide; (iii) a fragment of (i) or (ii) that codes for a fragment of the polypeptide OrfY that has at least part of the promoter activity-regulating activity of the OrfY polypeptide; and (iv) a sequence that is complementary to any of (i) to (iii). Included in such nucleotide sequences is a sequence that, as the coding sequence, comprises a sequence selected from the group consisting of: (i) orfY as shown in Table 2 herein, and (ii) a fragment hereof that codes for a polypeptide having the promoter activity-regulating activity of the intact OrfY polypeptide.

In further aspects the invention provides a vector comprising a nucleotide sequence as defined above and a cell transformed with such a vector.

In yet another aspect there is provided a method of regulating in a cell the activity of a promoter sequence, the activity of which is regulatable by the OrfY polypeptide or a fragment hereof having the promoter activity-regulating activity of the intact OrfY polypeptide, the method comprising inserting into the cell the nucleotide sequence as defined above and combining it with appropriate expression signals to permit the expression of the coding sequence of said sequence, resulting in the production of an OrfY polypeptide or a polypeptide that is at least 15% identical with OrfY, or a fragment hereof having at least part of the promoter activity-regulating activity of OrfY.

In yet a further aspect the invention provides a method of producing a desired gene product, the method comprising constructing a cell that comprises the nucleotide sequence or the vector as defined above, and a sequence coding for the desired gene product, said coding sequence is under the control of a promoter, the activity of which is regulatable by the OrfY polypeptide or a polypeptide that is at least 15% identical with OrfY and has at least part of the promoter activity-regulating activity of OrfY, or a fragment hereof, cultivating the cell under conditions where both of (i) the sequence coding for the OrfY polypeptide, the polypeptide that is at least 15% identical with OrfY and has at least part of the promoter activity-regulating activity of OrfY, or the fragment hereof having a least part of the promoter activity-regulating activity of the intact OrfY polypeptide and (ii) the sequence coding for the desired gene product are expressed, to obtain a biomass of cells, and harvesting the cells and/or the gene product.

The cells of the invention, in particular lactic acid bacteria as defined above, are useful in the manufacturing of food or feed products and as production strains in the industrial production of any desired gene products. Accordingly, the invention pertains in still further aspects to the use of a cell as defined above in a starter culture for manufacturing of a food product or a feed product and to the use of such a cell in a process of manufacturing a biologically and/or pharmaceutically active product and the use of the cell to deliver a biologically or pharmaceutically active product contained in the cell.

### DETAILED DISCLOSURE OF THE INVENTION

One primary objective of the present invention is to provide an improved regulatable gene expression system, in particular such an improved system that is useful in lactic acid bacteria. The provision of such improved systems is based on the discovery in Lactococcus lactis of an assumingly trans-acting regulatory polypeptide, OrfY, the absence of which results in a dramatic decrease of the activity of an inducible promoter including a promoter the activity of which is regulated by pH. Over-expression of orfY resulted in a significant increase in expression from an inducible promotor.

In one aspect of the invention there is provided an isolated nucleotide sequence that comprises a coding sequence for this identified promoter activity-regulating polypeptide. As used herein the expression “promoter activity-regulating” implies both an enhancement of promoter activity and a reduction in promoter activity. When the polypeptide has an enhancing effect, it is also referred to as a promoter activator. The regulatory effect of the polypeptide of the invention may be exerted on both inducible and constitutive promoters.

Additionally, the invention pertains to a nucleotide sequence coding for a promoter activity-regulating polypeptide that is at least 15% identical to the sequence coding for OrfY and that has at least part of the promoter activity-regulating activity of the OrfY polypeptide. Also included are such sequences that code for a polypeptide having at least 20%, such as at least 30%, 40%, 50% or even at least 60% identity with the OrfY polypeptide. It is contemplated that such coding sequences showing a high degree of identity with the coding sequence for OrfY can be identified in lactic acid bacterial species including any of such species mentioned above, as well as in other organisms including other gram-positive bacterial species and gram-negative bacterial species.

Alternatively, the coding sequence is a fragment of a sequence coding for the OrfY polypeptide or the sequence coding for a polypeptide being at least 15%, 20%, 30%, 40%, 50% or 60% identical with OrfY, said fragment hereof at least partially having the promoter activity-regulating activity of the OrfY polypeptide. The nucleotide sequence of the invention may also include a coding sequence that is complementary to any of the above coding sequences including DNA and RNA sequences.

In specific embodiments the nucleotide sequence of the invention comprises, as the coding sequence, the open reading frame, orfY as shown in Table 2 hereinbelow or a fragment hereof that codes for a polypeptide having at least part of the promoter activity-regulating activity of the intact OrfY polypeptide.

In useful embodiments the nucleotide sequence of the invention is one that further comprises a promoter region comprising at least one promoter sequence, the activity of which is regulatable by the OrfY polypeptide or a fragment hereof having the promoter activity-regulating activity of the intact OrfY polypeptide. In this context the term “promoter” is used in the conventional sense to designate the site whereto RNA polymerase can be bound.

The promoter sequence, the activity of which is regulatable by the OrfY polypeptide or a fragment hereof or a polypeptide being at least 15% identical herewith as defined above, may, in accordance with the invention be derived from any bacterial cell, but in preferred embodiments it is derived from a lactic acid bacterial species including the above species and Bifidobacterium spp. In useful embodiments, the promoter sequence is derived from a promoter region of Lactococcus lactis including Lactococcus lactis subspecies lactis, e.g the strain designated MG1363 (this strain is also referred to in the literature as Lactococcus lactis subspecies cremoris) and Lactococcus lactis subspecies lactis biovar. diacetylactis. Naturally occurring inducible promoter sequences which can be included in the nucleotide sequence of the invention may be isolated by any conventional methods for identifying and isolating nucleotide sequences comprising a promoter sequence and sequences having an effect on the activity of the promoter. Examples of promoter sequences which are useful in the present invention include the regulatable promoters disclosed in WO 94/16086 and WO 98/10079, including a region comprising the promoter P170. Typically, such a promoter sequence has a size which is in the range of 50 to 10,000 base pairs, such as in the range of 50 to 2000 base pairs including a range of 50 to 200 base pairs.

Preferably, the above promoter sequence of lactic acid bacterial origin is regulatable by physical or chemical factors or environmental or growth condition factors including pH, the growth temperature, the oxygen content, a temperature shift eliciting the expression of heat chock genes, the composition of the growth medium including the ionic strength/NaCI content, the presence/absence of essential cell constituents or precursors herefor, the accumulation of metabolites intracellularly or in the medium, the growth phase of the bacterium or the growth rate of the bacterium.

As stated above, orfY may control a group of promoters and their corresponding genes. Such a control may be illustrated by over-expression of orfY and subsequent evaluation of the promotor activity e.g. by expression of the corresponding gene. This approach is illustrated hereinafter in the examples where over-expression of orfY increases production of a promotor (P170) controlled extracellular nuclease and intracellular β-galactosidase. Other promoters controlled by orfY could be identified by using different techniques. One example is to use two-dimensional gel electrophoresis to analyze protein expression in a wildtype L. lactis MG1363 strain and compare the resulting protein pattern to the protein pattern obtained in an isogenic orfY mutant strain. Protein spots, which are missing in the orfY mutant strain would most likely represent gene products that are controlled by orfY. These protein spots can be excised from the gel, digested with trypsin to generate unique sets of peptides for each protein. The masses of the peptides can be determined by e.g. matrix assisted laser desorption ionization-time of flight (MALDI-TOF) mass spectroscopy. The peptide fingerprint can be used for searching in e.g. the Mascot database (http://www.matrixscience.com) and will secure a unique identification of proteins that are under control of the orfY regulator in L. lactis. To demonstrate that orfY actually regulates the expression of the identified proteins on the transcriptional level, a traditional transcriptional analysis (Northern blot analysis or reporter-gene fusions) of each gene should be performed, or alternatively DNA-chip technology could be used for simultaneously analysis of all the identified genes. In this case, the genes encoding proteins under orfY control can be amplified by PCR and spotted on a DNA-chip. Total RNA from the wildtype strain and the orfY mutant strain is extracted, reverse transcribed in the presence of two different fluorescent dyes, which becomes incorporated into the cDNA. The two labeled cDNA populations are subsequently hybridized to the DNA-chip and the hybridization signals are detected by fluorescent scanning of the DNA-chip. Genes on the DNA-chip that require orfY for transcription will only hybridize to the cDNA population isolated from the wildtype strain. Based on the complete genome sequence of L. lactis, the promoters located upstream of these genes can easily be cloned and analyzed as has been performed for the P170 promoter.

It will be understood that when the promoter sequence is one, the induction or regulation of which is controlled by one or more substances present in a conventional growth medium, substances which are not normally components of such media, such as antibiotics or bacteriocins are, in accordance with the invention, generally not included as environmental or growth condition factors.

In addition to the promoter sequence that is regulatable by the OrfY polypeptide or fragments hereof or polypeptides or fragments showing at least 15% identity as defined above with OrfY, the nucleotide sequence of the invention may comprise at least one further nucleotide sequence element the presence of which has a regulatory effect on the expression of a gene operably linked to the promoter region being regulated. As used herein, the expression “further nucleotide sequence” may include a sequence encoding a ribosome binding site, a transcription factor binding site, a repressor binding site, a site mediating attenuated or autoregulated gene expression, a DNA sequence which can be transcribed into mRNA having an altered affinity for the ribosome or an altered affinity for nucleases, a DNA sequence comprising a transcription terminus, or any other sequence capable of modulating and/or enhancing gene expression. In the present context, this term will also include DNA sequences in the promoter sequence region which has no specifically recognised function, such as e.g. sequences located between or adjacent to −10 and −35 promoter sequences and other consensus sequences.

Also encompassed by the invention are the promoter sequences naturally associated with the sequence coding for a promoter activity-regulating polypeptide as defined herein including the promoter sequence for the orfY gene as shown in Table 2 below. The use of such promoter sequences to direct the expression of coding sequences with which such promoter sequences are not naturally associated is contemplated.

It is also within the scope of the invention to operably link the coding sequence for the promoter activity-regulating polypeptide or fragment with at least one promoter with which the coding sequence for the polypeptide or fragment is not naturally associated, ie a foreign promoter. Such a promoter may be selected from a constitutive promoter and a regulatable/inducible promoter including promoters as described above.

In another useful embodiment, the nucleotide sequence according to invention comprises a promoter regulating, at the transcriptional or the translational level, the expression of any of the above sequences coding for a promoter activity-regulating polypeptide or fragment hereof. Such a promoter may be a promoter that is naturally associated with such coding sequences or it is, alternatively, a promoter not naturally associated with said sequences. In accordance with the invention, the promoter regulating the expression of the promoter activity-regulating polypeptide or a fragment hereof is selected from a constitutive promoter and a regulatable, ie inducible promoter. Examples of promoters that is suitable for this purpose include the regulatable promoters of lactic acid bacterial origin that are disclosed in WO 94/16086 and WO 98/10079, respectively and derivatives or modifications hereof. When the promoter regulates the expression of the promoter activity-regulating gene polypeptides at the transcriptional level, it may be via affecting the structure or function of mRNA, e.g. resulting is a modulated folding hereof or in changed susceptibility to the effect of RNAses.

In a further aspect of the invention there is provided a vector or replicon comprising the nucleotide sequence as defined above including a plasmid, a bacteriophage, a transposable element or a cosmid.

There is also provided a cell transformed with the above nucleotide sequence or with the above vector. Any type of transformable cells is contemplated including bacterial cells of gram-positive and gram-negative species, fungal cells such as of filamentous fungi, yeast cells, plant cells, animal cells including human cells. A particularly interesting transformed cell is a bacterial cell selected from the group consisting of lactic acid bacterial species including a Lactococcus species.

The present invention provides, as it is mentioned above, in yet another aspect a method of regulating in a cell, including the above transformed cells, the activity of a promoter sequence, the activity of which is regulatable by the OrfY polypeptide or a homologue of the OrfY polypeptide, or fragments hereof having the promoter activity-regulating activity of the intact OrfY polypeptide. This method comprises the steps of inserting in the cell the nucleotide sequence of the invention that codes for the OrfY polypeptide, a fragment hereof or a polypeptide having the above-defined identity with OrfY and combining it with appropriate expression signals to permit the expression of the coding sequence of said sequence, resulting in the production of an OrfY polypeptide or a fragment hereof having promoter regulating activity. In one embodiment, the promoter sequence that is to be regulated is a promoter sequence naturally occurring in the cell. Alternatively, the promoter sequence, the activity of which is to be regulated is a sequence not naturally occurring in the cell. In preferred embodiments, the promoter sequence to be regulated is of lactic acid bacterial origin such a the P170 promoter as described in the following.

In accordance with the invention, the promoter sequence, the activity of which is to be regulated is selected from the group consisting of a regulatable promoter and a constitutive promoter including regulatable promoters that are regulated by a factor selected from the group consisting of pH, the growth temperature, the oxygen content, a temperature shift eliciting the expression of heat chock genes, the composition of the growth medium including the ionic strength/NaCI content, the presence/absence of essential cell constituents or precursors herefor, the accumulation of metabolites intracellularly or in the medium, the growth phase of the cell and the growth rate of the cell.

In a presently preferred embodiment, the activity of the promoter sequence that is to be regulated is enhanced by the expression of the sequences coding for the OrfY polypeptide or the polypeptide that is at least 15% identical herewith, or the fragment hereof. The enhancement effect is reflected in a higher level of expression of the gene the expression of which is controlled by the promoter sequence. The level of such an enhancement may be substantial in cells that do not contain a sequence coding for the promoter activity-regulating polypeptide or fragment, e.g. at least 1-fold including at least 5-, 10- 50- 100- or 200-fold. When the cell naturally contains such a coding sequence, but is transformed with 1 or more additional copies of the coding sequence, the enhancement of promoter activity should result in at least 10% increase of the expression level of the gene under control of the promoter sequence, the activity of which is regulated, such as at least 50%, 100% or 200% increase of expression level. It is also contemplated that the OrfY polypeptide and homologues or fragments hereof may also have an inhibiting effect on promoter activity resulting in a reduced expression level of the gene under control of the promoter sequence, the activity of which is regulated by the OrfY polypeptide and homologues or fragments hereof.

In a still further aspect there is provided a method of producing a desired gene product, the method comprising constructing a cell that comprises (i) a nucleotide sequence as defined above that codes for the OrfY polypeptide or a polypeptide that is at least 15% identical hereto as it is defined above or fragments hereof, having at least part of the promoter activity-regulating activity of the OrfY polypeptide or the above vector comprising such nucleotide sequences, and (ii) a sequence coding for the desired gene product, said coding sequence is under the control of a promoter, the activity of which is regulatable by the OrfY polypeptide or a fragment hereof, cultivating the cell under conditions where both of (i) the sequence coding for the OrfY polypeptide or the fragment hereof having at least part of the promoter regulating activity of the intact OrfY polypeptide and (ii) the sequence coding for the desired gene product are expressed, and harvesting the resulting cells or the gene product.

The cell as defined above is preferably selected from gram-positive or gram-negative bacterial cells, fungal cells, yeast cells, plant cells, animal cells including human cells. Presently preferred gram-positive cells are cells selected from the group consisting of lactic acid bacterial species including cells of a Lactococcus species. In the above method of producing gene products, the promoter sequence, the activity of which is regulated by the OrfY polypeptide, homologue or fragment is a promoter sequence as defined above.

The cells of the invention and in which the activity of least one promoter sequence is regulated by the OrfY polypeptide or homologues or fragments hereof as defined herein are useful in starter cultures for manufacturing of a food product or a feed product, in particular starter cultures of lactic acid bacteria. The cells of the invention are also useful in processes of manufacturing desired gene products including enzymes such as aspartic proteases and pharmaceutically active products such as vaccine components. Additionally, the cells are as such useful as vehicles for biologically and/or pharmaceutically active gene products.

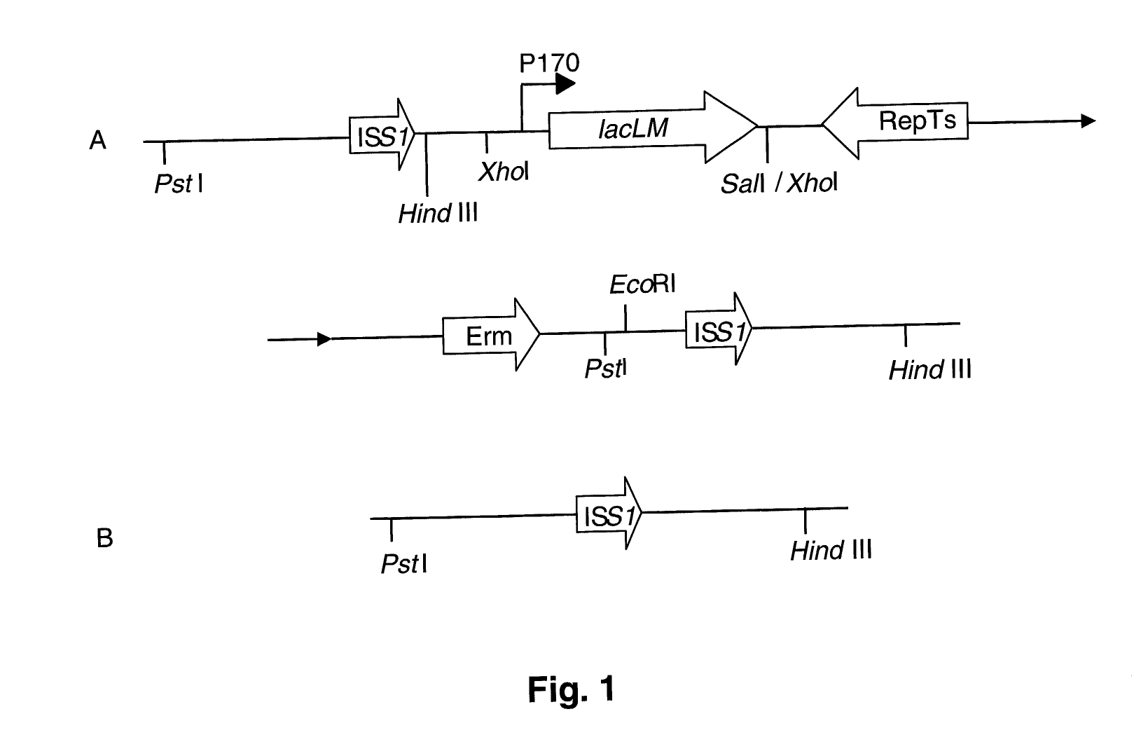

The invention will now be described in further details in the below examples and the figures wherein FIG. 1 shows the chromosomal structure after pSMBI 20 transposon mutagenesis. The line upstream and downstream of the ISS1 indicates the chromosomal DNA, which is interrupted by the transposon. pSMBI 20 transposon insertion leads to integration of the vector plasmid sequence between the duplicated ISS1 sequences. The P170-lacLM gene cassette, the temperature sensitive replicon (RepTs), and the erythromycin resistance marker (Erm) are shown. Relevant restriction sites used in the study are marked. FIG. 1B illustrated the chromosomal structure after excision of pSMBI20, resulting in a food-grade ISS1 mutant. The figure is not drawn to scale,

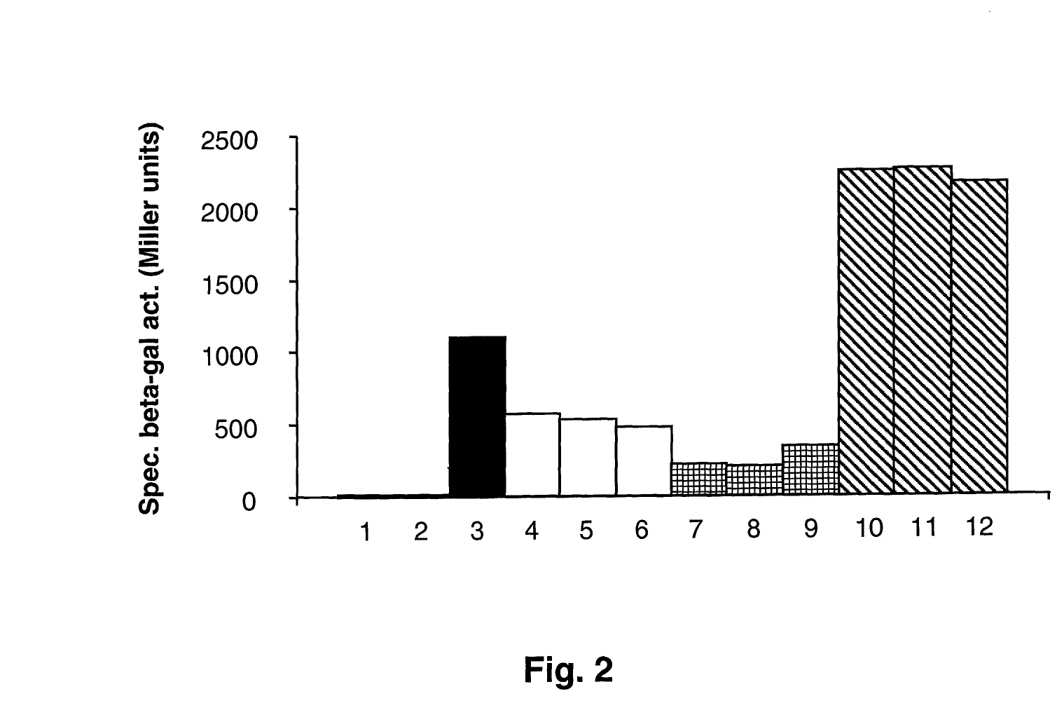

FIG. 2 illustrates measurement of specific β-galactosidase activity in GM17 medium. Bars 1-3, 4-6, 7-9 and 10-12, respectively show the β-galactosidase activity in strains containing plasmid pAMJ752, pSMBI25, pSMBI27 and pAMJ769, respectively. Bars 1, 4, 7 and 10 are the respective plasmids in the ISS1 mutant strain SMBI77. Bars 2, 5, 8 and 11 are the respective plasmids in the ISS1 mutant strain SMBI79. Bars 3, 6, 9 and 12 are the respective plasmids in the wild type strain MG1363,

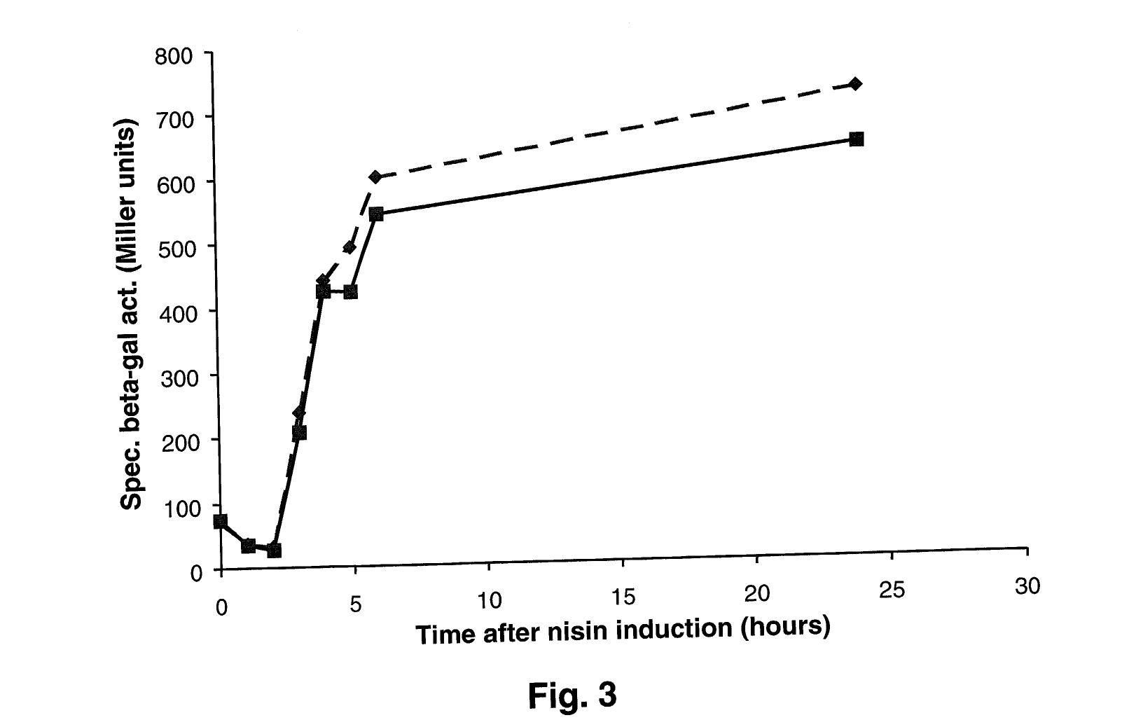

FIG. 3 shows the specific β-galactosidase activity versus time for strain SMBI145. Strain SMBI145 contains pNZ8010 (nisin inducible promoter) and pAMJ752 (lacLM fused to the P170 promoter). At time zero the culture was divided into two flasks and nisin was added to one flask (solid line) while the other was untreated (stippled line),

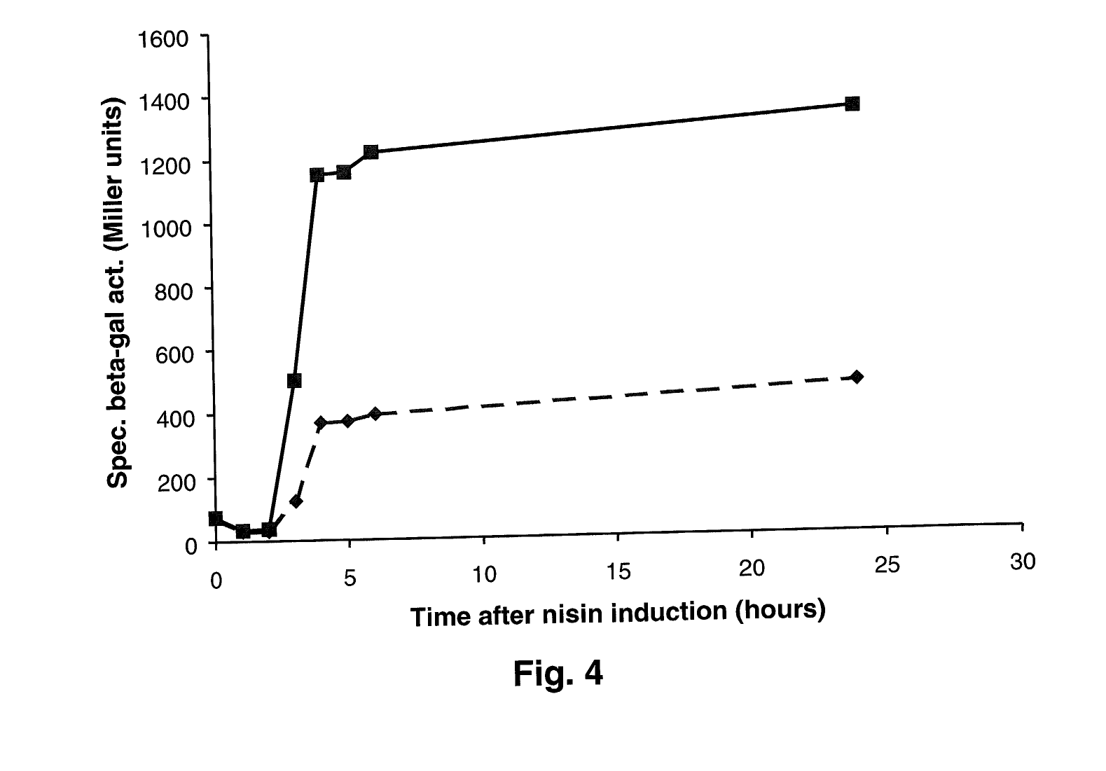

FIG. 4 shows the specific β-galactosidase activity versus time for strain SMBI143. Strain SMBI143 contains pSMBI137 (orfY fused to the nisin inducible promoter) and pAMJ752. At time zero the culture was divided into two flasks and nisin was added to one flask (solid line) while the other was untreated (stippled line),

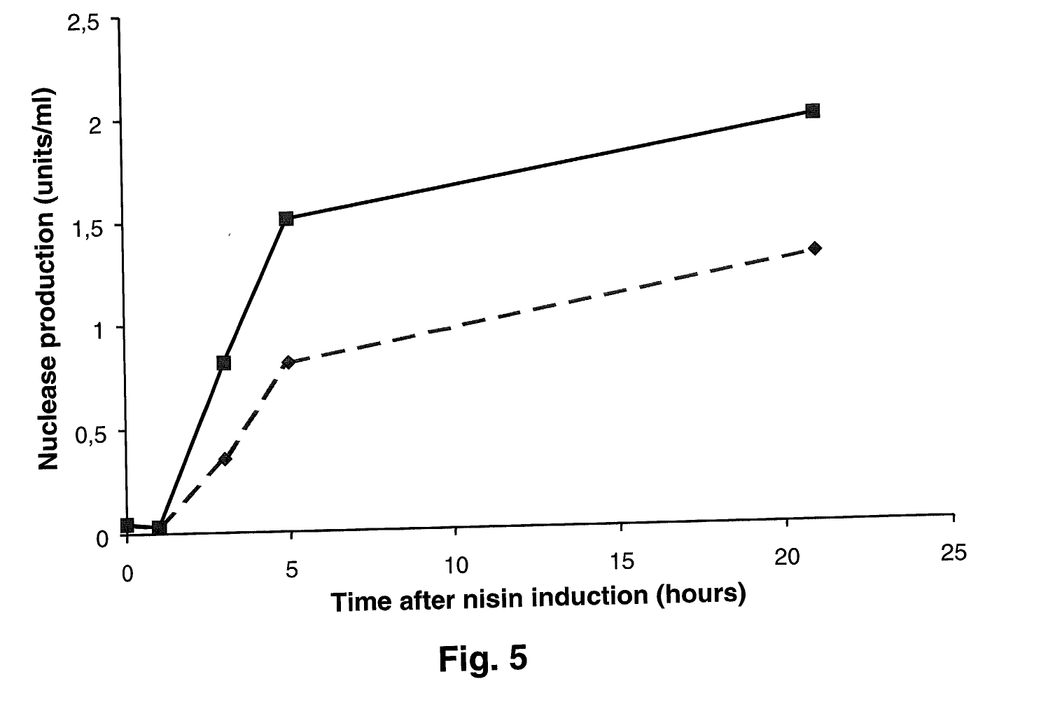

FIG. 5 shows the nuclease activity (units/ml) versus time for strain SMBI148. Strain SMBI148 contains pSMBI137 (orfY fused to the nisin inducible promoter) and p310mut2 (P170 fused to nucB). At time zero the culture was divided into two flasks and nisin was added to one flask (solid line) while the other was untreated (stippled line),

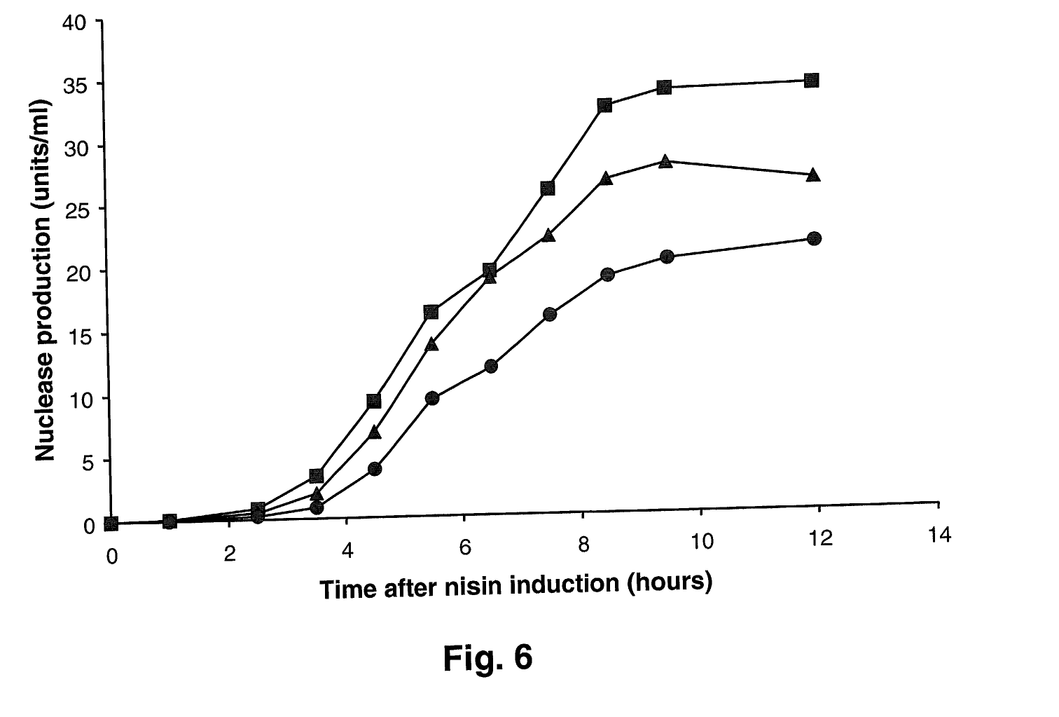

FIG. 6 shows the nuclease activity (units/mL) versus time for strain SMBI148 during inducing (squares) and un-inducing conditions (triangles), and in the control strain PRA294 after addition of nisin (circles). Strain SMBI148 contains pSMBI137 (orfY fused to he nisin inducible promoter) and p310mut2 (P170 fused to nucB), and

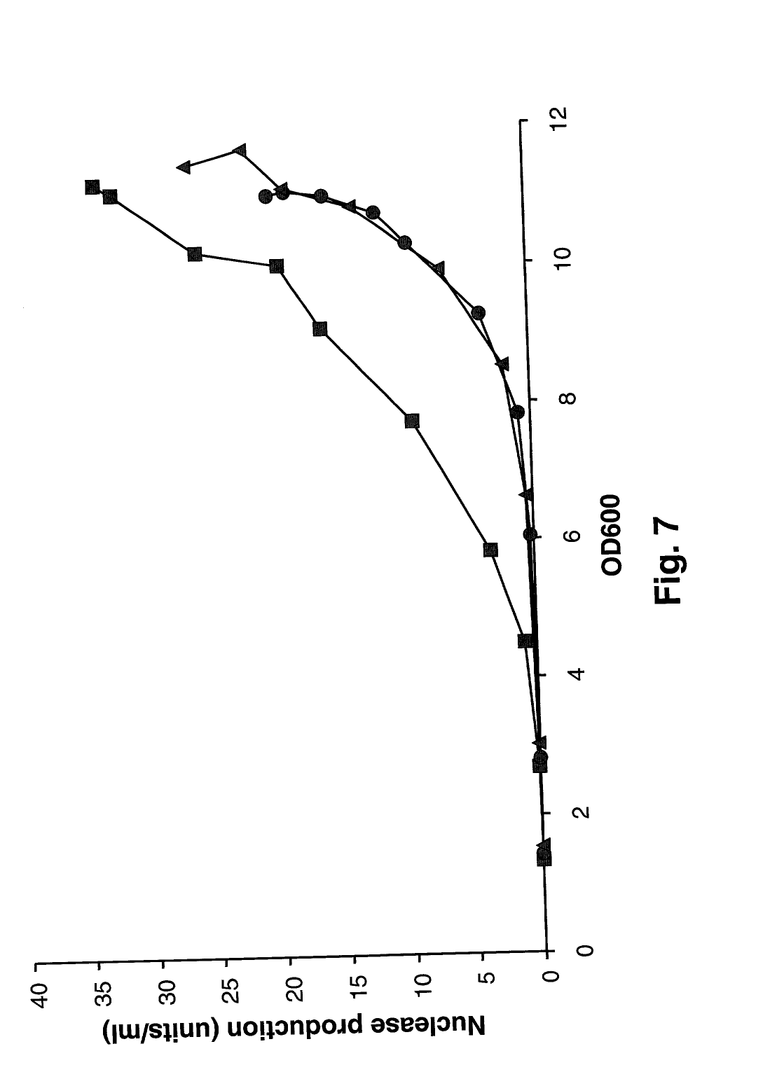

FIG. 7 shows the nuclease activity (units/mL) versus OD600 for strain SMBI148 during inducing (squares) and un-inducing conditions (triangles), and in the control strain PRA294 after addition of nisin (circles).

### EXAMPLES

Materials and Methods

Bacterial Strains, Plasmids and Growth Conditions

The bacterial strains and plasmids used in this experiment are listed in Table 1. E. coli strain DH10B (Grant et al., 1990) was grown in LB broth or LB agar at 37° C. L. lactis subsp. cremoris strain MG1363 (Gasson, 1983) was routinely grown at 30° C. in M17 (Oxoid) containing 0.5% glucose (GM17). The final pH was 5.5 after growth overnight in GM17. In experiments where a final pH 7.0 was required, a M17 medium containing 0.1% glucose and 0.1% arginine (ArgM17) was used. When required, erythromycin (erm) was added to a final concentration of 200 μg/ml and 1 μg/ml for E. coli and L. lactis, respectively. 5-Bromo-4-chloro-3-indolyl-β-D-galactopyranoside (Xgal) was used at 160 μg/ml in agar plates for L. lactis.

TABLE 1
Bacterial strains and plasmids used

| Bacteria or plasmids | Relevant characteristic(s) | Reference or source |
|---|---|---|
| Bacteria | | |
| L. lactis | | |
| MG1363 | Host for transposon mutagenesis and plasmid rescue (Erm^{S}) | Gasson, 1983 |
| Mut6 | MG1363::pSMBI20 transposon insertion mutant (Erm^{R}) | This study |
| Mut22 | MG1363::pSMBI20 transposon insertion mutant (Erm^{R}) | This study |
| Mut23 | MG1363::pSMBI20 transposon insertion mutant (Erm^{R}) | This study |
| SMBI77 | Mut6::ISS1 mutant (Erm^{S}) | This study |
| SMBI78 | Mut22::ISS1 mutant (Erm^{S}) | This study |
| SMBI79 | Mut23::ISS1 mutant (Erm^{S}) | This study |
| E. coli | | |
| DH10B | E. coli cloning host | Grant et al, 1990 |
| Plasmids | | |
| pGh9:ISS1 | Transposition plasmid (Erm^{R}) | Maguin et al, 1996 |
| pAMJ752 | Regulated P170 promoter from pAMJ752 inserted upstream of | Madsen et al, 1999 |
| | lacLM in pAK80 (Erm^{R}) | |
| pSMBI20 | P170-lacLM fragment from pAMJ752 inserted into pGh9:ISS1 (Erm^{R}) | This study |
| pSMBI25 | Constitutive P1 promoter from phage φ31 inserted upstream of | Chapter 5, this thesis |
| | lacLM in pAK80 (Erm^{R}) | |
| pSMBI27 | Constitutive P2 promoter from phage φ31 inserted upstream of | Chapter 5, this thesis |
| | lacLM in pAK80 (Erm^{R}) | |
| pAMJ769 | Constitutive P170 promoter from pAMJ769 inserted upstream of | Madsen et al, 1999 |
| | lacLM in pAK80 (Erm^{R}) | |
| pMut6-HindIII | HindIII rescue plasmid from Mut6 (Erm^{R}) | This study |
| pMut6-PstI | PstI rescue plasmid from Mut6 (Erm^{R}) | This study |
| pMut23-HindIII | HindIII rescue plasmid from Mut23 (Erm^{R}) | This study |
| pMut23-PstI | PstI rescue plasmid from Mut23 (Erm^{R}) | This study |

Measurement of β-galactosidase Activity

β-Gal assays were performed on cultures grown overnight as described in Israelsen et al., 1995. Measurements were averages of at least three independent experiments.

Cloning, Plasmid Isolation and Transformation

DNA manipulations were performed according to standard procedures (Sambrook et al., 1989). PCR amplifications were performed using the Taq DNA polymerase from Gibco BRL as recommended by the manufacturer. Plasmid DNA from E. coli was isolated using the Jet Prep columns (Genomed). Plasmid DNA from L. lactis was isolated as described (O'Sullivan and Klaenhammer, 1993b). Chromosomal L. lactis DNA was prepared as described in Johansen and Kibenich, 1992. E. coli competent cells (ElectroMAX DH10B™) were electroporated as described by the manufacturer and L. lactis was transformed using electroporation as described in Holo and Nes, 1989.

DNA Sequencing and Homology Search

Rescued plasmid DNA was sequenced using a Thermo Sequenase fluorescent-labelled primer cycle sequencing kit (Amersham), Cy5-labeled primers and an ALFexpress DNA sequencer (Pharmacia Biotech). The deduced amino acid sequence was analysed using the BlastP program available at www.ncbi.nlm.nih.gov/BLAST.

### EXAMPLE 1

Identification of a Putative P170 Transcriptional Factor

1.1. Construction of a Modified pGh9:ISS1 Transposon Vector

A modified pGh9:ISS1 transposon vector, pSMBI20, was constructed by insertion of a 4.1 kb XhoI-SalI fragment from pAMJ752 containing the P170-lacLM gene cassette (Madsen et al., 1999) into the unique XhoI site of pGh9:ISS1 (Maguin et al., 1996). pSMBI20 replicates at 28° C. in L. lactis and expression of the lacLM reporter gene is controlled by the P170 promoter. At 370° C. plasmid replication ceases and transposition is revealed by selection for erythromycin resistance at this non-permissive temperature.

1.2. pSMB120 Transposon Mutagenesis

Transposon mutagenesis with pSMBI20 was performed essentially as described in Maguin et al., 1996 with minor modifications as indicated below. pSMBI20 was transformed into L. lactis MG1363 and selection was performed on GM17 plates containing 160 μg/ml Xgal and 1 μg/ml erythromycin. A strain containing pSMBI20 was grown overnight in GM17 medium containing 1 μg/ml erythromycin at 28° C. The overnight culture was diluted 100-fold in GM17-1% NaCI medium without antibiotics and incubated for 150 minutes at 28° C. NaCI was added to adapt the culture to the subsequent increase in temperature. The culture was shifted to 37.50° C. and growth was allowed to continue for 150 minutes. Samples were diluted in 0.9% NaCI and plated at 37° C on GM17-1% NaCI plates containing 160 μg/ml Xgal and 1 μg/ml erythromycin and the same plates without erythromycin. The transposition frequency defined as the number of erythromycin resistant cells obtained at 37° C. divided by the total cell count obtained at 37° C. was estimated to 2-5%, which is in agreement with the number previously reported using pGh9:ISS1 in L. lactis MG1363 (Maguin et al., 1996). Transposon mutants showing a reduced intensity of blue colour on Xgal plates were isolated and analysed by PCR. This initial PCR screening was used for detection of a specific fragment covering the P170 promoter and the amino terminal of lacLM. Only transposon mutants that resulted in amplification of this fragment were analysed further while the remaining were discarded. Three transposon insertion mutants, Mut6, Mut22 and Mut23, which showed the expected PCR fragment, were identified.

1.3. Isolation of ISS1 Mutants by Excision of the Transposed Vector

The three mutants were grown overnight in GM17 medium without selection at 37° C. The cultures were subsequently diluted 10⁶-fold in the same medium and incubated at 28° C. for 24 hours. This step permits plasmid replication resulting in turn in homologous recombination between the flanking ISS1 sequences. The cultures were diluted and plated onto GM17 plates without erythromycin at 37° C. During this step plasmid replication should cease resulting in plasmid loss when cells are plated without selection for erythromycin. Colonies were tested for loss of the erythromycin resistance marker by streaking on GM17 plates containing erythromycin. More than 80% of the colonies were found to be erythromycin sensitive. Using this protocol, the transposed vector sequence was excised from Mut6, Mut22, and Mut23 resulting in SMBI77, SMBI78, and SMBI79, respectively.

1.4. Cloning and Sequencing of the DNA Sequences Adjacent to the Transposon Insertion Points

The DNA sequences located adjacent to the duplicated ISS1 sequences of the pSMBI20 transposon insertion mutants strains, Mut6 and Mut23, were rescued by isolation of genomic DNA, digestion with HindIII or PstI, followed by religation and transformation into L. lactis strain MG1363. Rescue of the DNA sequence adjacent to ISS1 insertion points of Mut6 resulted in the two plasmids pMut6-HindIII and pMut6-PstI, while pMut23-HindIII and pMut23-PstI were obtained by plasmid rescue from Mut23. Plasmids obtained by PstI rescue were sequenced using the Cy5 labelled ISS1.F1 primer, 5′ GGA ACG CTC TTC GGA TTT TCG GTA TC 3′ (SEQ ID NO: 1), and plasmids obtained by HindIII rescue were sequenced using the Cy5 labelled ISS1.R1 primer, 5′ GTT CAT TGA TAT ATC CTC GCT 19 GTC 3′ (SEQ ID NO: 2). The nucleotide sequence described in this experiment has been deposited in the EMBL database under the accession number AJ278292.

1.5 Identification of a Putative P170 Specific Transcription Factor by ISS1 Transposon Mutagenesis of L. lactis MG 1363

To identify genes affecting P170 transcription, the temperature sensitive pGh:ISS1 transposition system (Maguin et al., 1996) was used. This system has previously been used successfully to identify genes involved in acid resistance and DNA repair of L. lactis (Duwat et al., 1997; Rallu et al., 2000). First, pGh9:ISS1 was modified to permit easy identification of mutants showing altered P170 transcription after transposon mutagenesis (FIG. 1A). This was done by the insertion of a DNA fragment, which contained the P170-lacLM gene cassette (Madsen et al., 1999), into pGh9:ISS1 resulting in pSMBI 20. It was assumed that random transposition of pSMBI 20 into the chromosome of L. lactis would result in blue colonies on plates containing Xgal, if the function of the targeted genes does not affect P170 transcription. In contrast, pale blue or white colonies were expected when genes necessary for P170 transcription were inactivated by the transposon insertion.

Transformation of L. lactis MG1363 with pSMBI20 resulted in exclusively blue colonies when selection was performed on GM17 plates containing Xgal at the permissive temperature of 28° C., demonstrating that all cells contained a structurally stable plasmid expressing the β-galactosidase reporter gene. Subsequently, the transposition protocol was used for generation of a collection of about 10,000 transposon insertional mutants, which were selected on GM17 plates containing Xgal and erythromycin. This permitted identification of mutants with an altered expression of the lacLM reporter gene indicative of a change in P170 promoter activity. A total of 34 clones that showed reduced intensity of blue colour on GM17-Xgal-erm plates (white to pale blue) were selected. The initial analysis of these clones by PCR revealed that only three clones showed the expected pattern, indicating that rearrangements had occurred in the remaining clones probably causing the altered phenotype. The three clones showed a pale blue (Mut6 and Mut22) or white (Mut23) phenotype on Xgal plates. This indicated that one or more genes involved in the positive regulation of P170 transcription was/were inactivated in the three clones.

1.6 Generation of Stable ISS1 Mutants by Excision of the Transposed Vector

To ascertain that the transposon insertions indeed affect the transcriptional activity of the P170 promoter, excision of the pGh9:ISS1 sequence was carried out to generate mutant strains that only contained a single ISS1 copy at the affected loci. The transposon insertion mechanism of ISS1 leads to integration of the transposition vector between the duplicated ISS1 sequences. This structure is maintained at 37° C., but if the temperature is decreased to 28° C. plasmid replication will be initiated and homologous recombination between the ISS1 elements will occur, resulting in excision of the plasmid sequence from the chromosome, leaving a single ISS1 element at the affected locus (FIG. 1B). Excision of the pSMBI20 vector sequence resulted in the erythromycin sensitive mutant strains SMBI77, SMBI78 and SMBI79, respectively.

### EXAMPLE 2

Analysis of P170 Expression in the ISS1 Mutant Strains

To analyse the activity of the P170 promoter in the three ISS1 mutants obtained in Example 1, the strains were transformed with plasmid pAMJ752 containing the strongest P170 derivative transcriptionally fused to the lacLM reporter gene of the promoter probe vector pAK80 (Israelsen et al., 1995). Determination of β-galactosidase activity was subsequently performed on cultures, which were grown overnight in GM17 medium (FIG. 2).

The wild type L. lactis strain MG1363 containing plasmid pAMJ752 was used as a control. The β-galactosidase activity in strain SMBI78/pAMJ752 was identical to the activity obtained in the wild type strain MG1363/pAMJ752 indicating that a rearrangement, which was not detected by the PCR analysis, had occurred in this strain. Therefore, this strain was not analysed further. In contrast, the β-galactosidase activity in the two strains SMBI77/pAMJ752 (˜-12 Miller units, FIG. 2, Bar 1) and SMBI79/pAMJ752 (˜-12 Miller units, FIG. 2, Bar 2) was reduced to about 1% of the level obtained in the wild type strain MG1363/pAMJ752 (˜-1100 Miller units, FIG. 2, Bar 3). This clearly shows that one or more genes involved in P170 transcription was/were affected by the transposition.

To confirm that the observed reduction in β-galactosidase activity was caused by inactivation of a regulatory P170 specific gene and not by inactivation of a gene that affects promoter activity in general, the β-galactosidase expression of three different constitutive promoters was also examined. Two constitutive phage promoters contained in pSMBI25 (Bars 4-6 in FIG. 2) and pSMBI27 (Bars 7-9 in FIG. 2) and the constitutive P170 variant contained in pAMJ769 (Bars 10-12 in FIG. 2) were introduced into the two mutant strains and the β-galactosidase activity was compared with the wild type strain containing the same plasmids. The β-galactosidase expression in the three plasmids containing the constitutive promoters was similar in both mutant strains and the wild type strain. These results confirm that the inactivated loci were specific for P170 directed transcription.

In the following, the gene that was inactivated by pSMBI20 is termed orfY.

The effect of pH on expression in the mutant strain SMBI77 containing pAMJ752 was analysed by measuring the β-galactosidase expression in ArgM17 medium. The β-galactosidase activity obtained in this medium was 6 Miller units, which corresponds to 50% of the activity obtained in GM17 medium (˜12 Miller units). In comparison the β-galactosidase activity in the wild type strain MG1363 containing pAMJ752 decreases from 1,100 Miller units in GM17 medium to 17 Miller units in ArgM17 medium. This shows that inactivation of orfY has a tremendous impact on the P170-directed expression during growth in a medium resulting in low pH (induced conditions), while the expression is less affected in a medium resulting in neutral pH (repressed conditions). These results indicate that the transcriptional activity of P170 in ArgM17 medium is due to a background level, probably caused by binding of the RNA polymerase complex at the extended -10 region even in the absence of orfY. However, orfY still seems to have a minor effect on the expression level in ArgM17 medium as the β-galactosidase activity decreases from 17 Miller units in the wild type strain to 6 Miller units in the mutant strains. This could possibly reflect that the low expression of orfY that occurs in the wild type strain in ArgM17 medium is sufficient to increase the P170 directed expression level about three-fold compared to the same conditions in the mutant strain. Interestingly, the regulation of P170-directed expression in the mutant strain is still regulated two-fold by pH (expression in GM17 versus ArgM17). This could indicate that additional factors could be involved in the regulation or that other factors might compensate the defect in orfY.

### EXAMPLE 3

Sequencing of orfY and Homology Searches for OrfY, its Derived Gene Product

3.1. Sequencing of orfY and its Possibly Regulatory Sequences

Using the plasmid rescue facility of pSMBI 20, the DNA regions flanking the transposon insertion sites in the two mutants Mut6 and Mut23 were cloned. The nucleotide sequences located upstream and downstream, respectively of the transposon insertion points were subsequently determined from the rescued plasmids and assembled into the DNA sequence presented in the below Table 2 (SEQ ID NO: 3) that shows the nucleotide sequence of the orfY gene of L. lactis. The amino acid sequence encoded by the orfY gene is also shown in Table 2 (SEQ ID NO: 4). The extended −10 promoter sequences located upstream of orfY and upstream of the putative ribose 5-phosphate isomerase gene homologue are marked in bold letters. The putative translation initiation codon of orfY is underlined. The putative regulatory inverted repeat upstream of orfY and the putative transcriptional terminator downstream of orfY are indicated with vertical arrows. Triangles mark the position of PSMBI 20 transposon insertion sites in Mut6 and Mut23, respectively.

TABLE 2

| | |
|---|---|
| 1 | ATAGTGCGCCAGCAGTTGTAATTGTTTGGATAATATACTATCTTATTCACGGTAAACATC |
| | -10 |
| 61 | AAAAATCAAGCTCTGAAGTTTGATTTAAATGATTTACATAAAACATGTTATAATAAAGGG |
| | ————————→ ← |
| 121 | GTTACAGCCCTGTATATGGCGAAATAAATGAATAAAAAATAGCGAGTAGATGAGTTTTAA |
| | ORF Y M S F K |
| 181 | AATGAAAGAAATGGCAAACGTAAACATTGAATATCTAATCAATACACTGGAACAAAAAAA |
| | M K E M A N V N I E Y L I N T L E Q K K |
| 241 | AGTGAGTGTTGTAACACGCAAAAAACATAGTTATATCATGTATCAAGGGATTGAATCAGA |
| | V S V V T R K K H S Y I M Y Q G I E S E |
| 301 | ATATATCTATGTACTCAAAGATGGTGTAGCGAAGATTAGCAATATTTTAAGAGATGGTCG |
| | Y I Y V L K D G V A K I S N I L R D G R |
| 361 | TGAATTTAATATTGCTTATGTTGCGGAGCCAGACTTTGTTTCTTTATTGGAAGAGAAACA |
| | E F N I A Y V A E P D F V S L L E E K Q |
| 421 | AAACGATGGAATTTCAGCATTATTTAATGTACGAATTGAGTCTCCAACAGCCAGTTTTTA |
| | N D G I S A L F N V R I E S P T A S F Y |
| 481 | CAAAATTTCACGCAGTGATTTTTGGAATTGGGTTCGTGAGGATTTGAATTTATTCAGAGT |
| | K I S R S D F W N W V R E D L N L F R V |
| 541 | TGTTGATGACTTTTATAAACGAAGACTAGCACTTAATTTAGAAATTCTTCAAAAGATGAC |
| | V D D F Y K R R L A L N L E I L Q K M T |
| 601 | AATCAATGGTAAGAAGGGAGCGGTTTGCGCTTGCCTTCACAGTTTGATTGATGATTTCGG |
| | I N G K K G A V C A C L H S L I D D F G |
| 661 | AATAAGAAAAAAAGATGGAATTCTGATTGATTTTACCGTCACTAATGAAGATATTGCAGG |
| | I R K K D G I L I D F T V T N E D I A G |
| | Mut6 ▾ ▾Mut23 |
| 721 | TTTTTGTGGTATTTCTACACGAAATAGTGTTAACCGTATTCTTCATGATTTAAAGGATGA |
| | F C G I S T R N S V N R I L H D L K D E |
| 781 | AAAAGTAATTGGAGTGATTGATAATAAAATTATGATTTATAATCCTCAATACTTAGAAGA |
| | K V I G V I D N K I M I Y N P Q Y L E E |
| | -10 |
| 841 | ATATATTAGTTAATATAAATAAATAAAAAAGCTACTTTAAGTAGCTTTTTTGCTATAAT |
| | ———————————→ ←——————————— |
| | Y I S * |

The sequence analysis shows that transposon insertions had occurred in an open reading frame, OrfY, of 227 codons encoding a putative 26.3 kDa protein. The same open reading frame was interrupted in both of mutants Mut6 and Mut23, but the integration sites were separated by two base pairs. No obvious ribosome binding site was identified upstream of the translation initiation codon, indicating a low level of translation. An extended −10 promoter region (5′ TGTTATAAT 3′) was identified 56 upstream of the translation initiation codon, but no consensus −35 sequence was identified in the expected position. This is analogous to the situation of the P170 promoter and other regulated promoters from L. lactis (Sanders et al., 1998b; Walker and Klaenhammer, 1998; Madsen et al., 1999) and indicates that expression of orfY also is regulated. Interestingly, an inverted repeat is located 20 bp upstream of the extended −10 region, suggesting that this structure may play a role in gene expression. Alternatively, this structure might serve as a transcriptional terminator of an upstream located gene. Downstream of orfY is another inverted repeat [ΔG(25° C.)=−8.8 kcal mol⁻¹], which might serve as a rho-independent transcription terminator. The presence of a putative promoter and a putative transcription terminator indicates that orfY is transcribed as a single unit in L. lactis.

Distally from orfY, another open reading frame highly homologous to ribose 5-phosphate isomerases from a variety of organisms was identified. This gene is also preceded by an extended −10 promoter, but lacks a consensus −35 sequence. The same genomic organisation has been identified in the fully sequenced L. lactis strain IL1403 (Bolotin et al., 1999). The two genes were located close to position 2,300,000 on the genome map of strain IL1403. The presence of the ribose 5-phosphate isomerase homologue, which most likely is transcribed from its own promoter, supports the assumption that orfY is not part of an operon structure. These data strongly support that the reduced transcriptional activity of P170 is due to inactivation of orfY and rules out the possibility that polar effects are responsible for the reduced of P170 promoter activity.

3.2. The P170 Activator Shows Homology to a fnr Gene from Bacillus licheniformis

Homology search using the BlastP program revealed that OrfY was 27% identical to a FNR-like protein from Bacillus licheniformis. The results are summarised in the below Table 3 that shows a comparison of OrfY from L. lactis (SEQ ID NO: 5) and FNR from B. licheniformis (SEQ ID NO: 6). Identical amino acids are boxed. The four putative cysteines involved in iron binding in the FNR protein from B. licheniformis are shaded. The three cysteines in OrfY are also shaded.

[image]

It has previously been demonstrated that FNR of B. licheniformis functions as an oxygen-sensitive transcriptional regulator (Klinger et al., 1998). In general, OrfY showed the highest identity to a group of proteins belonging to the CRP-FNR family of global regulators. This type of proteins controls the expression of overlapping modulons in response to glucose starvation and anaerobic respiration/metabolism in e.g. E coli (reviewed by Guest et al., 1996). FNR of E. coli contains a cysteine rich N-terminal domain, which may be part of an iron-binding redox-sensing domain while the DNA binding motif of FNR is associated with a helix-turn-helix motif located in the C-terminal. In contrast, the FNR proteins of B. licheniformis and B. subtilis contain clusters of cysteine residues in the C-terminal end of the protein (Klinger et al., 1998; Cruz et al., 1995). Despite this structural difference in the FNR proteins from the Bacillus species and E. coli, the mechanism of activation by oxygen is likely to be conserved. Three C-terminal cysteines residues were identified in OrfY but only one cysteine residue was conserved relative to the FNR protein of B. licheniformis. However, this cysteine in FNR of B. licheniformis is not predicted to be part of the iron-binding domain.

Three FNR-like proteins (FLP) have recently been identified in Lactobacillus casei (FLP) Irvine and Guest, 1993; Gostick et al., 1998) and L. lactis (FlpA and FlpB) (Gostick et al., 1999; Scott et al, 2000). FLP of L. casei has a homo-dimer structure in which each subunit can form an intramolecular disulphide bond. It has been proposed that FLP controls gene expression of target genes/operons by a redox-mediated transcriptional switch in which the active DNA-binding form is the oxidised form (disulphide) of FLP. The disulphide form of FLP binds to the site (C^{A}/_{C}TGA-N₄-TCA^{G}/_{T}G) (SEQ ID NO: 7). In contrast, the properties of FlpA of L. lactis resemble FNR of E. coli more than FLP of L. casei by binding to a FNR site (TTGAT-N₄-ATCAA) (SEQ ID NO: 8) but not to a FLP site. Furthermore, FlpA was unable to form the intramolecular disulphide bond, which is the active form of FLP.

Presently, it seems unlikely that orfY activates and regulates gene expression similarly to the mechanisms described for FLPs or FNRs. Using a transcriptional fusion of P170 to the promoter less β-galactosidase reporter gene of pAK80, the expression of P170 in both aerobic and anaerobic cultures was analysed. However, no obvious difference in production of β-galactosidase was observed indicating that the oxygen levels do not affect P170-directed transcription.

Recently, the pfl gene encoding the pyruvate-formate-lyase (PFL) of L. lactis has been identified (Arnau et al., 1997). Expression of PFL in both E. coli and L. lactis increases during anaerobic conditions and this induction is FNR dependent in E. coli. Upstream of the pfl promoter of L. lactis two putative FNR binding sites were identified (Arnau et al., 1997). However, no data has yet demonstrated the FNR dependence on PFL expression in L. lactis. To analyse the activity of the pfl promoter, we therefore transformed the orfY mutant strain as well as the wild type strain with a plasmid containing a fragment with the putative FNR sites flanking the pfl promoter, which was transcriptionally fused to the lacLM reporter gene of pAK80. However, measurement of β-galactosidase activity in the two orfY-mutant strains did not show any difference, indicating that orfY does not affect the transcriptional activity of the pfl promoter of L. lactis.

OrfY did not show any significant homology to the three FLPs identified in L. casei and L. lactis (data not shown). The conserved position of the cysteine residues of the three FLPs was not identified in OrfY and the well-conserved ES-R motif seen in FLP and FNR proteins was not present in OrfY. No FNR or FLP binding sites were detected on the 27 bp cis-acting fragment that is required for P170 regulated expression. No obvious helix-turn-helix motif was identified in OrfY, which indicates that OrfY does not regulate P170 expression by protein-DNA interactions in the promoter region. The influence of orfY on P170 promoter activity could therefore be indirect by regulating the expression of a second factor, which is responsible for control of P170 expression. Alternatively, OrfY could interact with the RNA-polymerase complex through protein-protein interactions thereby increasing the affinity for e.g. the P170 promoter.

### EXAMPLE 4

Cloning of orfY into the Nisin Controlled Expression Vector, pNZ8010.

The following examples were conducted in order to analyse whether over-expression of orfY could increase the production of different reporter gene products directed by a inducible promotor. The gene encoding orfY from Lactococcus lactis strain MG1363 was PCR amplified using the primers FNR-8-BamHI (5′ TAG TAG GAT CCG AAA GGA GGC ACT CAA AAT GAG TTT TAA AAT GAA AGA AAT GGC 3′) (BamHI site underlined) and FNR-2-BamHI (5′ TAG TAG GAT CCG AAT ATT TCG ATA TCA CGC TGA C 3′) (BamHI site underlined). The 795 bp PCR fragment was ligated into the pCR2.1 vector (Invitrogen) resulting in plasmid pSMBI136. pSMBI136 was transformed into E. coli strain DH10B selecting for ampicillin resistance. The DNA sequence of orfY in pSMBI136 was confirmed. pSMBI136 was subsequently digested with BamHI and the DNA fragment containing the orfY gene was inserted into BamHI digested pNZ8010 (de Ruyter et al., 1996) resulting in pSMBI137. pSMBI137 was transformed into E. coli selecting for chloramphenicol resistance. The correct orientation of orfY relative to the nisin inducible promoter was verified by DNA sequencing. This promoter is tightly regulated, i.e. not active in the absence of nisin and gradually active in a dose-dependent manner in the presence of nisin in strains containing the nisRK genes.

Over-Expression of orfY Increases the Production of a P170 Controlled Intracellular β-galactosidase in Flask Culture.

L. lactis strain NZ9000 (Kuipers et al., 1998) is a L. lactis MG1363 derived strain that contains a copy of the nisRK genes on the chromosome allowing nisin-induced expression from pNZ8010 derived plasmids. To analyze the effect of induced over-expression of orfY on P170 gene expression, we transformed pSMBI137 into strain NZ9000 that also harboured plasmid pAMJ752. Plasmid pAMJ752 contains the strongest pH and growth phase inducible promoter, P170, transcriptionally fused to the lacLM reporter-genes (Madsen et al., 1999). Strain NZ9000, containing the two plasmids pSMBI137 and pAMJ752 was named SMBI143. A NZ9000 derived strain named SMBI145 containing the two plasmids pNZ8010 (no orfY gene inserted) and pAMJ752, served as a control in the expression studies.

The two L. lactis strains SMBI143 and SMBI145 were grown separately overnight in flasks in GM17 medium supplemented with 5 μg/ml of chloramphenicol and 1 μg/ml erythromycin. The two cultures were subsequently diluted 100 times in fresh GM17 medium supplied with appropriate antibiotics and grown until OD600 reached approximately 0.3. The two cultures were subsequently divided into two new flasks. For each strain nisin was added to one flask to induce orfY expression while the other flask was left un-induced. Nisin (2.5% pure nisin, Sigma N5764) was used at a concentration corresponding to 0.1 ng/ml pure nisin. Culture samples were taken during growth for measurement of OD600. Culture samples for determination of β-galactosidase activity were taken at time zero (immediately before nisin induction) and then 1, 2, 3, 4, 5, 6 hours after induction and finally after induction overnight (24 hours). The specific β-galactosidase activity versus time after nisin induction obtained for the two strains, SMBI145 and SMBI143 is shown in FIGS. 3 and 4, respectively. As expected, the course of β-galactosidase expression was obviously nisin independent in strain SMBI145. This shows that pNZ8010 as such does not affect the P170 controlled β-galactosidase expression. Both the induced and the un-induced cultures of SMBI145 reached β-galactosidase around 600-700 Miller units after growth overnight. In contrast, a clear nisin dependent expression of β-galactosidase was observed in strain SMBI143. The un-induced culture reached approximately 500 Miller units after growth overnight while the nisin induced culture reached about 1300 Miller units after induction and growth overnight. Previously, we have described that expression levels around 1200 Miller units was obtained for pAMJ752 in L. lactis strain MG1363. In this study, we found that pAMJ752 only gave expression levels around 600-700 Miller units in the NZ9000 strain background. This indicates that even though the two strains are isogenic the presence of nisRK genes in the genome of NZ9000 apparently reduces the expression level compared to the expression level in strain MG1363. However, the herein described example clearly shows that over-expression of orfY has a significant effect on the production level of a P170 controlled intracellular reporter-gene product. Approximately 2-3 fold more β-galactosidase was obtained when orfY was co-expressed using the nisin inducible expression system. We also showed that over-expression of orfY did not have any effect on the β-galactosidase expression directed by the constitutive P170 promoter variant contained in plasmid pAMJ769 (data not shown). This indicates that over-expression of orfY does not have an effect on promoter activity in general, but that orfY over-expression is specific for the P170 promoter and possibly also on other promoters that could be part of an orfY controlled regulon.

Over-Expression of orfY Increases the Production of a P170 Controlled Extracellular Nuclease in Flask Culture.

The effect of orfY over-expression on a secreted gene product was also analyzed. This was done by the construction of a L. lactis strain, SMBI148, which contains the two plasmids pSMBI137 (nisA promoter expressing orfY) and p310mut2. p310mut2 contains the P170 promoter transcriptionally fused to the optimized signal peptide SP310 mut2 which in turn is translationally fused to the Staphylococcus aureus reporter gene nucB (PCT/DK00/00437). As a control we used the strain PRA294, which contains the two plasmids p310mut2 and pNZ8010. Similarly to the experiment described above, SMBI148 and PRA294 were grown overnight in flasks in GM17 medium supplemented with 5 μg/ml of chloramphenicol and 1 μg/m erythromycin. The two cultures were diluted 100 times in fresh GM17 medium supplied with appropriate antibiotics and grown until OD600 reached approximately 0.3. The two cultures were subsequently divided into two new flasks. For each strain nisin was added to one flask to induce orfY expression while the other flask was left un-induced. Culture samples were taken during growth for measurement of OD600. Culture samples for determination of nuclease activity were taken at time zero (immediately before nisin induction) and then 1, 3, 5 hours after induction and, finally, after induction overnight (23 hours). The nuclease activity versus time for the orfY over-producing strain, SMBI148, during induced and un-induced conditions is shown in FIG. 5. The production of secreted nuclease is clearly increased when orfY is over-produced. In the un-induced culture approximately 1.3 units/mL of secreted nuclease was obtained after induction and growth overnight. In contrast, 2.0 units/mL of secreted nuclease was found at the same time point when orfY was over-expressed using the nisin inducible promoter. Measurement of nuclease activity in the control strain PRA294 showed approximately 1.2 units/mL of secreted nuclease after overnight conditions (data not shown). This level was as expected nisin independent and corresponds to the un-induced level obtained in strain SMBI148 at the same time point. The result described in this example clearly shows that over-expression of orfY also has a significant effect on the production level of a P170 controlled extracellular reporter-gene product.

In the above example a nisin controlled promoter was used to over-produce the P170 regulator orfY. However, the over-production of orfY is certainly not restricted to the use of the nisin controlled promoter. Use of other inducible promoters as well as constitutive promoters for orfY over-expression are expected to have similar effects on the P170 promoter. In these studies, the orfY regulator was expressed on a plasmid, which is compatible with the plasmid carrying the P170 promoter and the reporter-gene system. Other possibilities includes e.g. replacment of the native promoter upstream of the chromosomal copy of orfY with a strong inducible or constitutive promoter. Use of a gene replacement strategy could be applied to achieve this construction (Leenhouts et al., 1991). Another option is to co-express orfY on the plasmid that also carries the P170 promoter. Again, orfY expression is not restricted to a specific promoter, but could be controlled by a variety of different promoters.

### EXAMPLE 5

Over-expression of orfY Increases the Production of a P170 Controlled Nuclease in a Batch Fermentation Process; in Particular when P170 is Located on a Medium-Copy Number Plasmid.

In the previous experiments, the effect of over-expression of orfY on P170 promoter activity was analysed in flasks containing GM17 medium. This type of set-up does not fully exploit the potential of the P170 promoter for production of recombinant proteins in L. lactis. To investigate whether orfY over-expression also affects the protein production in a process where P170 promoter activity is at optimal performance, we analyzed the effect of orfY (over-expression on the P170 promoter activity in a defined medium during a batch fermentation process.

In the following example the effect of orfY over-expression on a secreted gene product is described. For this analysis we used the two strains SMBI 148 (nisA promoter expresses orfY) and PRA294 (no orfY expression). Furthermore, we analyzed whether orfY over-expression also has an effect on P170 promoter activity, when P170 is located on a high-copy number plasmid. For this purpose a new strain, SMBI 158, was constructed. SMBI 158 is a NZ9000 derived strain and contains pSMBI 137 (nisA promoter expresses orfY) and pSMBI91 (patent application 25501 US 02). Plasmid pSMBI91 includes the strongest P170 variant (P170 promoter in plasmid pAMJ752) fused to the SP310 mut2-nucB gene cassette on a high-copy number plasmid. As control strain SMBI160 was used. SMBI160 contains the two plasmids pNZ8010 and pSMBI91.

The four strains SMBI148, PRA294, SMBI158 and SMBI160 were grown overnight in GM17 medium supplemented with 5 μg/mL of chloramphenicol and 1 μg/mL erythromycin. The strains SMBI148 and SMBI158 were each inoculated in two fermentors each containing LM5-50 medium and appropriate antibiotics. The control strains PRA294 and SMBI160 were each inoculated in one fermentor, which also contained LM5-50 medium and appropriate antibiotics. pH was kept at 6.5 by automatic addition of 4.5 M potassium hydroxide and the temperature was kept at 30° C. The final cell density in LM5-50 medium is approximately 10. Expression of orfY in strain SMBI148 (medium-copy number plasmid) was induced at OD600≈1.3 by addition of 0.1 ng/mL nisin. No nisin was added to the second fermentor containing SMBI148. The same amount of nisin (0.1 ng/mL) was added to the fermentor with the control strain PRA294 at OD600≈1.4. Samples were taken during the course of fermentation for measurement of OD600 and determination of nuclease activity. FIG. 6 shows the nuclease activity versus time for strain SMBI148 during inducing and un-inducing conditions and the nuclease activity versus time for the control strain PRA294 (nisin added). The production of the nuclease is clearly increased when orfY is over-expressed in strain SMBI148. After nisin induction approximately 34 units/mL of secreted nucleased was reached, while the un-induced level reached approximately 28 units/mL. The nuclease activity in the control strain, PRA294, reached approximately 22 units/mL. These results indicate that the nisin promoter is somewhat leaky in this experiment. The un-induced level of nuclease activity in strain SMBI148 is 27% higher than that obtained in the control strain PRA294, which only contains the chromosomal copy of orfY. The effect of orfY over-expression is obvious when the induced level of nuclease activity in strain SMBI148 is compared to the level obtained in strain PRA294. In this case the nuclease activity is approximately 55% higher in strain SMBI148. In FIG. 7, the nuclease activity is plotted versus OD600. From FIG. 7 it is noticed that the P170 directed production of nuclease take place at lower cell density when orfY is over-expressed (SMBI148 induced culture) compared to the case observed in strain SMBI148 (un-induced culture) and PRA294 (nisin added, no orfY gene expressed). The kinetics of nuclease production was almost identical in the un-induced culture of strain SMBI148 and the culture of strain PRA294 to which nisin was added.

Similarly, orfY over-expression in strain SMBI158 (high-copy number plasmid) was induced at OD600≈1.1 and the other fermentor containing SMBI1158 was left un-induced. Nisin was added to the control strain SMBI160 at OD≈1.3. Samples were subsequently taken during the course of fermentation for measurement of OD600 and determination of nuclease activity. Over-expression of orfY did only have a slight effect on the P170 promoter activity, when P170 is located on a high-copy number plasmid. Approximately 47 units/mL of secreted nuclease was obtained 11.5 hours after nisin induction in strain SMBI158. The un-induced culture of SMBI158 reached approximately 43 units/mL of nuclease while the control strain SMBI160 reached approximately 40 units/mL nuclease 11.5 hours after addition of nisin. In conclusion, orfY only seems to have a minor effect on the promoter when P170 is carried on a high-copy number plasmid compared to a medium-copy number plasmid. This could reflect bottlenecks in the translational machinery or the secretion apparatus, meaning that increased transcription from the P170 does not per se result in concomitant increased translation or secretion of the nuclease gene product.

### REFERENCES

Arnau J, Jørgensen F, Madsen S M, Vrang A and Israelsen H (1997). Cloning, expression and characterization of the Lactococcus lactis pfl gene, encoding pyruvate formate-lyase. J. Bacteriol. 179:5884-5891.

Cruz R H, Boursier L, Moszer I, Kunst F, Danchin A and Glaser P (1995). Anaerobic transcription activation in Bacillus subtilis: identification of distinct FNR-dependent and-independent regulatory mechanisms. EMBO J. 14:5984-5994.

deRuyter P G, Kuipers O P, Meijer W C and de Vos W M (1996). Controlled gene expression systems for Lactococcus lactis with food-grade inducer nisin. Appl. Environ. Microbiol. 62:3662-3667.

deRuyter P G, Kuipers O P, Meijer W C and de Vos W M (1997). Food-grade controlled lysis of Lactococcus lactis for accelerated cheese ripening. Nat. Biotechnol. 15:976-979.

Djordjevic G M and Klaenhammer T R (1998). Inducible gene expression systems in Lactococcus lactis. Mol. Biotechnol. 9:127-139.

Duwat P, Cochu A, Ehrlich S D and Gruss A (1997). Characterization of Lactococcus lactis UV-sensitive mutants obtained by ISS1 transposition. J. Bacteriol. 179:4473-4479.

Gasson M J (1983) Plasmid complements of Streptococcus lactis NCDO712 and other lactic acid streptococci after protoplast-induced curing. J. Bacteriol. 154:1-9.

Grant S G, Jesse J, Bloom F R and Hanahan D (1990). Differential plasmid rescue from transgenic mouse DNAs into Escherichia coli methylation-restriction mutants. Proc. Natl. Acad. Sci USA. 87:4645-4649.

Gostick D O, Green J, Irvine A S, Gasson M J and Guest J R (1998). A novel regulatory switch mediated by the FNR-like protein of Lactobacillus casei. Microbiology. 144:705-717.

Gostick D O, Griffith H G, Shearman C A, Scott C, Green J, Gasson M J and Guest J R (1999). Two operons that encode FNR-like proteins in Lactococcus lactis. Mol. Microbiol. 31:1523-1535.

Guest J R, Green J, Alistair S, Spiro I and Spiro S (1996). The FNR Modulon and FNR-Regulated Gene Expression. In: Regulation of Gene Expression in Escherichia coli. Lin ECC and Lynch AS (eds.) R G Landes Company, pp. 317-337.

Holo H and Ness I F (1989). High-Frequency Transformation, by Electroporation, of Lactococcus lactis subsp. cremoris Grown with Glycine in Osmolytically Stabilized Media. Appl. Environ. Microbiol. 55:3119-3123.

Hols P, Klereresbezem M, Schank A N, Ferain T, Hugenholtz J, Delcour J and deVos W M (1999). Conversion of Lactococcus lactis from homolactic to homoalanine fermentation through metabolic engineering. Nat. Biotechnol. 17:588-592.

Irvine A S and Guest J R (1993). Lactobacillus casei contains a member of the CRP-FNR family. Nucleic Acids Res. 21:753.

Israelsen H, Maden S M, Vrang A, Hansen E B and Johansen E (1995). Cloning and partial characterization of regulated promoters from Lactococcus lactis Tn917-lacZ integrants with the new promoter probe, pAK80. Appl. Environ. Microbiol. 61:2540-2547.

Johansen E and Kibenich A (1992). Characterization of Leuconostoc isolates from commercial mixed strain mesophilic starter cultures. J. Dairy Sci. 75:1186-1191.

Kleerebezem M, van Kranenburg R, Tuinier R, Boels I C, Zoon P, Looijesteijn E. Hugenholtz J and de Vos W M (1999). Exopolysaccharides produced by Lactococcus lactis: from genetic engineering to improved rheological characteristics? Antonie van Leeuwenhook. 76:357-365.

Klinger A, Schirawski J, Glaser P and Unden G (1998). The fnr gene of Bacillus licheniformis and the cysteine ligands of the C-terminal FeS cluster. J. Bacteriol. 180:3483-3485.

Kok J (1996). Inducible gene expression and environmentally regulated genes in lactic acid bacteria. Antonie van Leeuwenhook. 70:129-145.

Kuipers O P, Beerthuyzem M M, Siezen R J and de Vos W M (1993). Characterization of the nisin gene cluster nisABTCIPR of Lactococcus lactis. Requirement of expression of the nisA and nisi genes for development of immunity. Eur. J. Biochem. 216:281-291.

Kuipers, O P., de Ruyter, P. G. G. A., Kleerebezem, M., and de Vos, W. M. (1998) Quorum sensing-controlled gene expression in lactic acid bacteria. J. Biotechnol. 64:15-21.

Leenhouts, K., Kok, J., and Venema, G. (1991) Replacement recombination in Lactococcus lactis. J. Bacteriol. 173:4794-4798.

Madsen S M, Arnau J, Vrang A, Givskov M and Israelsen H (1999). Molecular characterization of the pH-inducible and growth phase-dependent promoter P170 of Lactococcus lactis. Mol. Microbiol. 32:75-87.

Maguin E, Prevost H, Ehrlich S D and Gruss A (1996). Efficient insertional mutagenesis in lactococcal and other gram-positive bacteria. J. Bacteriol. 178:931-935.

O'Sullivan D J and Klaenhammer T R (1993) Rapid Mini-Prep Isolation of High-Quality Plasmid DNA from Lactococcus and Lactobacillus spp. Appl. Environ. Microbiol. 59:2730-2733.

Rallu F, Gruss A, Ehrlich S D and Maguin E (2000). Acid- and multistress-resistant mutants of Lactococcus lactis: identification of intracellular stress signals. Mol. Microbiol. 35:517-528.

Sambrook J, Fritsch E F and Maniatis T (1989). Molecular cloning: A Laboratory Manual. Cold Spring Harbor Laboratory.

Sanders J W, Leenhouts K, Burghoorn J, Brands J R, Venema G and Kok J (1998). A chloride-inducible acid resistance mechanism in Lactococcus lactis and its regulation. Mol. Microbiol. 27:299-310.

Scott C, Guest J R and Green J (2000) Characterization of the Lactococcus lactis transcription factor FlpA and demonstration of an in vitro switch. Mol. Microbiol. 35:1383-1393.

Walker S A and Klaenhammer T R (1998). Molecular characterization of a phage-inducible middle promoter and its transcriptional activator from the lactococcal bacteriophage phi31. J. Bacteriol. 180:921-931.

Wells J M, Robinson K, Chamberlain L M, Schofield K M and Le Page R W (1996). Lactic acid bacteria as vaccine delivery vehicles. Antonie van Leeuwenhook 70:317-330.

1. An isolated nucleotide sequence comprising a coding sequence which comprises
(i) a sequence coding for the promoter activity-regulating polypeptide OrfY as defined herein;
(ii) a sequence coding for a polypeptide that is at least 15% identical to the sequence of (i) and that has at least part of the promoter activity-regulating activity of the OrfY polypeptide;
(iii) a fragment of (i) or (ii) that codes for a fragment of the polypeptide OrfY that has at least part of the promoter activity-regulating activity of the OrfY polypeptide; or
(iv) a sequence that is complementary to any of (i) to (iii).

2. A nucleotide sequence according to claim 1 comprising, as the coding sequence, a sequence which includes:
(i) orfY as shown in Table 2; or
(ii) a fragment thereof that codes for a polypeptide having at least part of the promoter activity-regulating activity of the intact OrfY polypeptide.

3. A nucleotide sequence according to claim 1 or 2 further comprising a promoter sequence the activity of which is regulatable by the OrfY polypeptide, the polypeptide that is at least 15% identical with OrfY, or a fragment thereof having at least part of the promoter activity-regulating activity of the intact OrfY polypeptide.

4. A nucleotide sequence according to claim 1 comprising a promoter regulating the expression of any of the coding sequences (i) to (iv) of claim 1 or the coding sequences (i) or (ii) of claim 2.

5. A nucleotide sequence according to claim 4 where the promoter regulating the expression of said coding sequences is a promoter not naturally associated with said sequences.

6. A vector comprising the nucleotide sequence of claims 1 or 2

7. A vector according to claim 6 which comprises a plasmid, a bacteriophage, a transposable element or a cosmid.

8. A cell transformed with the nucleotide sequence of claims 1 or 2 or the vector of claim 6.

9. A cell according to claim 8 which comprises a bacterial cell, a fungal cell, a yeast cell, a plant cell, an animal cell or a human cell.

10. A cell according to claim 9 which is a bacterial cell comprising lactic acid bacterial species.

11. A cell according to claim 10 which is of a Lactococcus species.

12. A method of regulating in a cell the activity of a promoter sequence, the activity of which is regulatable by the OrfY polypeptide or a polypeptide that is at least 15% identical with OrfY and having at least part of the promoter activity-regulating activity of OrfY, or a fragment thereof having at least part of the promoter activity-regulating activity of the intact OrfY polypeptide, the method comprising inserting into the cell the nucleotide sequence of claim 1 or 2 and combining it with appropriate expression signals to permit the expression of the coding sequence of said sequence, resulting in the production of an OrfY polypeptide, a polypeptide being at least 15% identical herewith, or a fragment thereof having at least part of the promoter activity-regulating activity of the OrfY polypeptide.

13. A method according to claim 12 wherein the promoter sequence is a promoter sequence naturally occurring in the cell.

14. A method according to claim 13 wherein the promoter sequence is a sequence not naturally occurring in the cell.

15. A method according to claim 14 wherein the promoter sequence is of lactic acid bacterial origin.

16. A method according to claim 15 wherein the promoter sequence is P170 as described hereinbefore.

17. A method according to claim 12 wherein the promoter sequence comprises a regulatable promoter promoter or a constitutive promoter.

18. A method according to claim 17 wherein the promoter sequence in the cell is a regulatable promoter regulated by a factor comprising pH, the growth temperature, the oxygen content, a temperature shift eliciting the expression of heat chock genes, the composition of the growth medium including the ionic strength/NaCI content, the presence/absence of essential cell constituents or precursors therefor, accumulation of a metabolite intracellularly or in the medium, the growth phase of the cell or the growth rate of the cell.

19. A method according to claim 18 wherein the cell is the cell of any of claims 8-11.

20. A method according to claim 12 wherein the activity of the promoter sequence is enhanced by the OrfY polypeptide or the fragment thereof.

21. A method of producing a desired gene product, the method comprising constructing a cell that comprises the nucleotide sequence of any of claims 1 or 2 or the vector of claim 6, and a sequence coding for the desired gene product, said coding sequence is under the control of a promoter, the activity of which is regulatable by the OrfY polypeptide or a polypeptide that is at least 15% identical with OrfY, or a fragment thereof having at least part of the promoter activity-regulating activity of OrfY, cultivating the cell under conditions where both of (i) the sequence coding for the OrfY polypeptide or a polypeptide that is at least 15% identical with OrfY, or the fragment thereof having at least part of the promoter activity-regulating activity of the intact OrfY polypeptide and (ii) the sequence coding for the desired gene product are expressed, and harvesting the resulting cells or the gene product.

22. A method according to claim 21 wherein the expression of the sequence coding for the OrfY polypeptide or the fragment thereof results in an enhancement of expression of the desired gene product.

23. A method according to claim 22 wherein the cell includes a bacterial cell, a fungal cell, a yeast cell, a plant cell, an animal cell or a human cell.

24. A method according to claim 23 wherein the cell is a bacterial cell comprising lactic acid bacterial species.

25. A method according to claim 24 wherein the cell is of a Lactococcus species.

26. A method according to claim 25 wherein the promoter sequence is a promoter sequence as defined in claim 13.

27. A method of manufacturing a food product or a feed product comprising:
culturing the food product or the feed product in a composition comprising a starter culture which includes a cell according to claim 8.

28. A method of manufacturing a pharmaceutically active product comprising:
formulating the pharmaceutically active product with a composition which includes a cell according to claim 8.

29. A method of delivering a biologically and/or pharmaceutically active gene product comprising:
inserting the biologically and/or pharmaceutically active gene product into a cell according to claim 8.
